(19) United States
(12) Patent Application Publication
(10) Pub. No.: US 2011/0201970 A1
(43) Pub. Date: Aug. 18, 2011

(54) METHOD AND APPARATUS FOR DIAGNOSING TEMPOROMANDIBULAR DISORDERS

(76) Inventor: James P. Boyd, SR., Rancho Santa Fe, CA (US)

(21) Appl. No.: 13/029,303

(22) Filed: Feb. 17, 2011

Related U.S. Application Data
(60) Provisional application No. 61/305,794, filed on Feb. 18, 2010.

(51) Int. Cl. A61B 5/107

(52) U.S. Cl. 600/590

(57) ABSTRACT

The present invention provides an improved method and apparatus for use in diagnosing temporomandibular disorders. The apparatus includes separate maxillary and mandibular appliances, which are sized and configured to fit over and be retained on a subject's incisors. The two appliances both include generally flat, T-shaped platforms that face each other in a confronting relationship for all possible mandibular movements, including excursive movement and protrusive/retrusive movement. When properly fitted on the subject's opposing incisors, the two appliances effectively prevent contact between the subject's opposing upper and lower teeth, including the posterior and canine teeth, and thereby reduce the intensity of clenching of the temporalis muscles. The apparatus thereby can be used to provide an inexpensive way for determining whether an intraoral discluder of this kind will be effective in relieving tension headaches, common migraine headaches, or other temporomandibular disorders.

### CROSS-REFERENCE TO RELATED APPLICATION

Priority is claimed to provisional patent application No. 61/305,794, filed Feb. 18, 2010, and entitled “An OTC Anti-Clenching Migraine Diagnosing Device.”

### BACKGROUND OF THE INVENTION

1. Field of the Invention

The present invention relates generally to intraoral devices and, more particularly, to intraoral discluders and related methods for use in relieving tension headaches, common migraine headaches, and temporomandibular disorders.

2. Background of the Invention

Tension and muscle-contraction headaches affect many people every day. The headaches often are recurring and, without effective treatment, can become very painful, restricting an individual's ability to think clearly and function effectively. The discomfort associated with tension and muscle-contraction headaches usually is due to pain from strained and fatigued muscles of the head. The majority of the muscles of the human head are not sufficiently strong to elicit the type of pain and discomfort associated with tension and muscle contraction headaches. However, that is not the case with the temporalis muscle, which is located on the side of the skull and extends from just behind the eye to just behind the ear, and which is an extremely powerful muscle that functions to close and elevate the jaw.

Under normal circumstances, the temporalis muscle should not exert a large static force by contracting isometrically, except possibly during normal chewing. Inappropriate isometric contraction of the temporalis muscle is commonly known as “clenching” and is clinically known as myofascial dysfunction. The intensity of the myofascial dysfunction varies according to the mandible's anterior/posterior position, with the intensity increasing as the mandible's position moves posteriorly. Unfortunately, myofascial dysfunction is particularly difficult to detect or diagnose, because the act of clenching is a relatively motionless act that is commonly done while a person is concentrating on another topic, or while sleeping.

As the muscular contraction condition of “clenching” continues, the muscle becomes fatigued and susceptible to spasming and cramping. The pain from spasming and cramping temporalis fibers can be severe, and it usually is diagnosed as a common migraine. Headache sufferers who seek the assistance of a physician typically are treated with muscle relaxants, analgesics, and/or physical therapy for the muscle fatigue. However, medications and physical therapy require continual treatment, and they treat only the symptoms of the underlying problem, not the source of the problem itself.

Headache sufferers who seek the assistance of a dentist, on the other hand, frequently are diagnosed with a temporomandibular disorder and treated with an intraoral “jaw-positioning” appliance. One such appliance is disclosed in U.S. Pat. No. 6,666,212, issued in the name of James P. Boyd, Sr. and entitled “Intraoral Discluder Device and Method for Preventing Migraine and Tension Headaches and Temporomandibular Disorders.” The appliance has two primary components, including (1) a prefabricated tray sized to fit over the wearer's maxillary or mandibular incisors, and (2) a quantity of an adaptable material disposed within the tray and adapted to conform to the shape of the incisors and thereby assist in retaining the appliance in its prescribed position. A third component has the form of protrusion, or platform, attached to the tray, for engagement with the opposing incisors. The platform is sized and configured to prevent contact between opposing upper and lower teeth, including the posterior and canine teeth and to thereby reduce the intensity of clenching of the temporalis muscles. The platform projects both forward and rearward of the incisors by a sufficient distance to ensure that it engages the opposing incisors when the mandible is in either a fully retrusive position or a fully protrusive position.

The U.S. Food and Drug Administration has approved this intraoral discluder for the “treatment of medically diagnosed migraine pain.” Nevertheless, physicians sometimes will hesitate to prescribe the discluders for their patients, because fitting them to a patient requires a separate visit to a specially trained dentist for treatment. This can be costly and time-consuming. Moreover, health care insurance companies typically refuse to cover the cost of the treatment, and patients frequently are reluctant to pay for the treatment themselves, because they cannot be certain that the discluder will fully alleviate their pain. No single treatment will help every sufferer. Physicians and patients, therefore, need some level of assurance that an intraoral discluder of this kind will be effective at treating their pain and its cost will be justified.

It should be apparent from the foregoing discussion, that there is a need for a simple and inexpensive approach for determining whether an intraoral discluder of the kind described above will be effective in relieving tension headaches, common migraine headaches, and temporomandibular disorders. The present invention satisfies this need.

### SUMMARY OF THE INVENTION

The present invention resides in an inexpensive intraoral apparatus, and a related method for using it, for diagnosing whether an intraoral discluder will be effective in relieving tension headaches, common migraine headaches, and temporomandibular disorders. The apparatus includes (1) a maxillary appliance including a tray that defines a channel sized to receive and be retained on a subject's maxillary incisors, and (2) a mandibular appliance including a tray that defines a channel sized to receive and be retained on the subject's mandibular incisors. The two appliances both further include platforms arranged in confronting relationship with each other when the appliances are retained on the subject's incisors. These platforms are sized and configured to ensure that they confront each other for all possible mandibular movements and positions, and to prevent contact between opposing upper and lower teeth, including the posterior and canine teeth. The apparatus thereby reduces the intensity of clenching of the subject's temporalis muscles.

In a more detailed features of the invention, the maxillary and mandibular appliances both further include a quantity of adaptable material disposed within the channels of their trays, to conformably adapt to the shape of the incisors and assist in retaining the appliances on the incisors. The adaptable material preferably is a curable material. In addition, the apparatus can further include an approximating device for temporarily fixing the two appliances in a prescribed relationship relative to each other while the appliances are disposed in the subject's mouth and the adaptable material cures. This approximating device can include a strip of adhesive tape that can be adhered to, and removed from, the maxillary and mandibular appliances.

In other more detailed features of the invention, the confronting surfaces of the platforms of the maxillary and mandibular appliances each are substantially smooth and slightly convex, such that they contact each other substantially tangentially for all possible mandibular movements and positions. These confronting surfaces preferably are generally T-shaped, each having a main section aligned with the incisors and a posterior section projecting in a posterior direction from a mid-portion of the main section.

In another more detailed feature of the invention, the maxillary and mandibular trays each include a front wall, a rear wall, and an interconnecting bottom (or top) wall, and these walls define the channel sized to receive and be retained on the subject's incisors. In addition, the apparatus can further include one or more leashes connecting the two appliance to each other. Each leash can include an elastic band connected at its ends to the maxillary tray and the mandibular tray, preferably at their front walls.

Other features and benefits of the invention should become apparent from the following description of the preferred embodiments, taken in conjunction with the accompanying drawings, which illustrate, by way of example, the principles of the invention.

### BRIEF DESCRIPTION OF THE DRAWINGS

FIG. 1 is a front perspective view of an intraoral apparatus including a maxillary appliance and a mandibular appliance, each having a channel sized to receive and be retained on a subject's respective maxillary and mandibular incisors.

FIG. 2 is a side elevational view of the maxillary and mandibular appliances of FIG. 1, shown with an approximating device in the form of a strip of adhesive tape, for temporarily securing the appliances in a prescribed relationship relative to each other.

FIG. 3 is a side elevational view of the maxillary and mandibular appliances of FIG. 2, shown being retained on the subject's respective maxillary and mandibular incisors by an adaptable material located with the channel of each appliance.

FIG. 4 is a schematic side elevational view of the maxillary and mandibular appliances of FIG. 3, showing the extreme positions of the mandibular appliance relative to the maxillary appliance for the maximum protrusive and retrusive positions of the subject's mandible.

FIGS. 5A and 5B are schematic front elevational views of the maxillary and mandibular appliances of FIG. 3, showing the extreme positions of the mandibular appliance relative to the maxillary appliance. FIG. 5A shows the maximum right excursive position, and FIG. 5B shows the maximum left excursive position.

FIGS. 6A-6E all relate to an alternative embodiment of the invention, in which the maxillary and mandibular appliances are connected to each other by two elastic leashes. In particular, FIG. 6A is a front elevation view of the appliances in a centered position; FIG. 6B is a front elevational view of the appliances in a fully excursive position relative to each other; FIG. 6C is a side elevational view of the appliances in a centered position; FIG. 6D is a side elevational view of the appliances with the mandibular appliance in a fully retrusive position; and FIG. 6E is a side elevational view of the appliances with the mandibular appliance in a fully protrusive position.

### DETAILED DESCRIPTION OF THE PREFERRED EMBODIMENTS

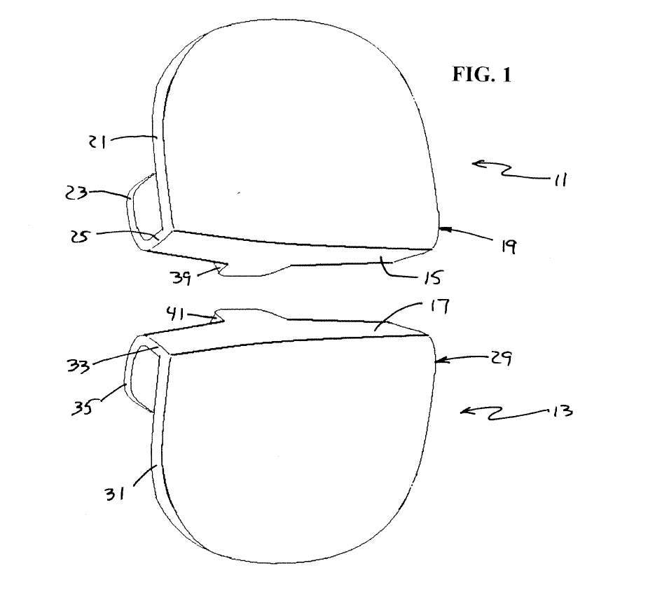

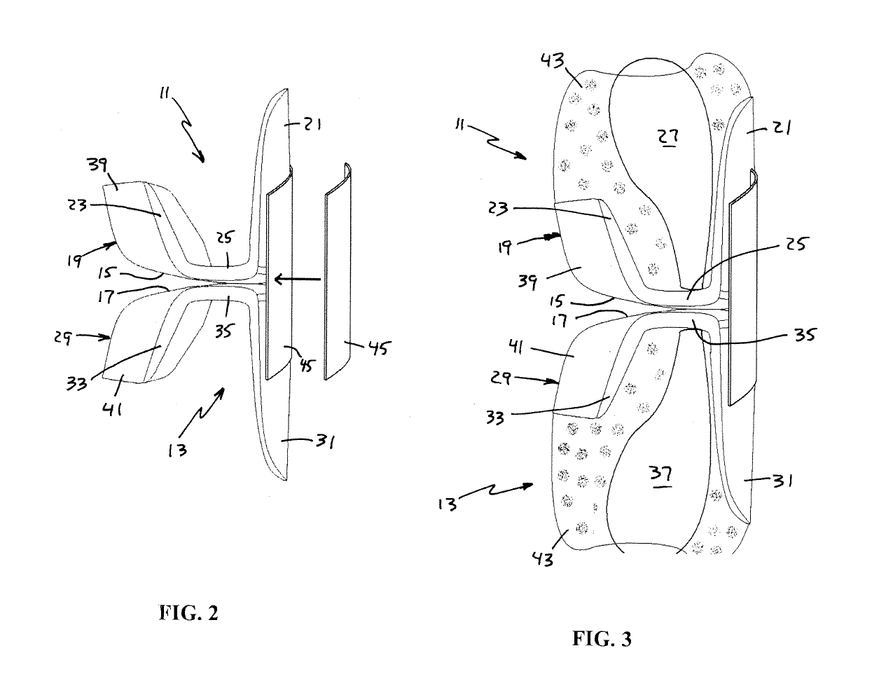

With reference now to the illustrative drawings, and particularly to FIGS. 1 and 2, there is shown an intraoral apparatus for use in diagnosing temporomandibular disorders of a subject. The apparatus includes a maxillary appliance 11 sized and configured to receive and be retained on the subject's maxillary incisors and a separate mandibular appliance 13 sized and configured to receive and be retained on the subject's mandibular incisors. The maxillary and mandibular appliances include T-shaped platforms 15 and 17, respectively, which are arranged to face each other in a confronting relationship. When properly fitted on the subject's opposing incisors, the two appliances effectively prevent any occlusive contact between opposing teeth. This not only reduces the intensity of clenching of the subject's temporalis muscles, but also avoids the need to involve a trained dental professional to ensure that individual teeth are not being traumatized by an improper occlusion. The apparatus thereby can be used to inexpensively determine whether an intraoral discluder of this kind will be effective in relieving tension headaches, common migraine headaches, or other temporomandibular disorders.

As best shown in FIGS. 2 and 3, the maxillary appliance 11 includes a tray 19 having a thin front wall 21, a thin rear wall 23, and a thin bottom wall 25 connecting the front and rear walls. Together, these three walls define a channel that is sized and configured to receive the subject's maxillary incisors 29. Similarly, the mandibular appliance 13 includes a tray 31 having a thin front wall 33, a thin rear wall 35, and a thin top wall 37 connecting the front and rear walls. Together, these three walls define a channel that is sized and configured to receive the subject's mandibular incisors 39. Preferably, each channel's length is sized to extend along two to four incisors. The trays preferably are made of a suitable intraoral dental material such as acrylic or polycarbonate.

The downward-facing surface of the bottom wall 25 of the maxillary tray 19 and the upward-facing surface of the top wall 35 of the mandibular tray 29 are configured to be smooth and slightly convex, and they are arranged in confronting relationship to each other when the appliances are installed on the subject's incisors. In addition, the maxillary appliance further includes a protrusion 39 projecting rearward from the mid-portion of the maxillary tray's rear wall 23, and the mandibular appliance similarly further includes a protrusion 41 projecting rearward from the mid-portion of the mandibular tray's rear wall 33. The downward-facing surface of the maxillary appliance's protrusion 39 blends smoothly with the downward-facing surface of the maxillary tray's bottom wall 25, thereby defining the generally T-shaped platform 15. Similarly, the upward-facing surface of the mandibular appliance's protrusion 41 blends smoothly with the upward-facing surface of the mandibular tray's top wall 35, thereby defining the generally T-shaped platform 17.

To facilitate retention of the maxillary tray 19 and mandibular tray 29 on the subject's respective maxillary incisors 27 and mandibular incisors 37, the appliances 11 and 13 further include a quantity of a suitable adaptable material 43 disposed within the channels of the two trays. This adaptable material conformably adapts to the shape of the incisors and assists in retaining the trays on the incisors. Examples of suitable adaptable material include silicone resins, polymers, enamels, rubbers, polycaprolactone, and any other material known to be used by dental practitioners for similar applications.

The maxillary appliance 11 and mandibular appliance 13 are installed on the subject's respective maxillary incisors 27 and mandibular incisors 37 by first placing a suitable amount of adaptable material 43 within the channels of the two trays 19, 29, and then placing the appliances in the subject's mouth, in alignment with the incisors. The subject then bites into adaptable material while positioning his jaw such that the trays are disposed with the T-shaped platforms 15, 17 contacting each other and with the trays' respective front walls 21, 31 in alignment. Holding this position is facilitated by temporarily adhering a strip of adhesive tape 45 to the front walls, spanning the interface between the two platforms. This is best shown in FIGS. 2 and 3. After the adaptable material has cured sufficiently to retain the general shape of the incisors, the tape can be removed and the appliances can be removed.

When the maxillary appliance 11 and the mandibular appliance 13 are subsequently used, they simply are placed individually on the subject's respective maxillary incisors 27 and mandibular incisors 37. The adaptable material 43 has a shape conforming with that of the incisors, but it is sufficiently resilient to allow the appliances to be readily snapped into place and subsequently to be readily removed.

When the appliances 11, 13 are installed on the subject's respective incisors 27, 37, their T-shaped platforms 15, 17 are arranged in confronting, or facing, relationship to each other. The confronting surfaces of the platforms are smooth and slightly convex and thus offer no significant resistance to mandibular movement, including both excursive (side-to-side) movement and protrusive/retrusive (forward/rearward) movement. In addition, the platforms are sized such that they remain in confronting relationship with each other throughout the full ranges of such movements. The slight convexity of the platforms' confronting surfaces ensures that the contact between them is substantially tangential for all possible positions. By ensuring that the only contact is between the two appliances themselves, the apparatus avoids the possibility of imposing any undue trauma to individual teeth and it thereby avoids the need for the fitting to be performed by a trained dental professional.

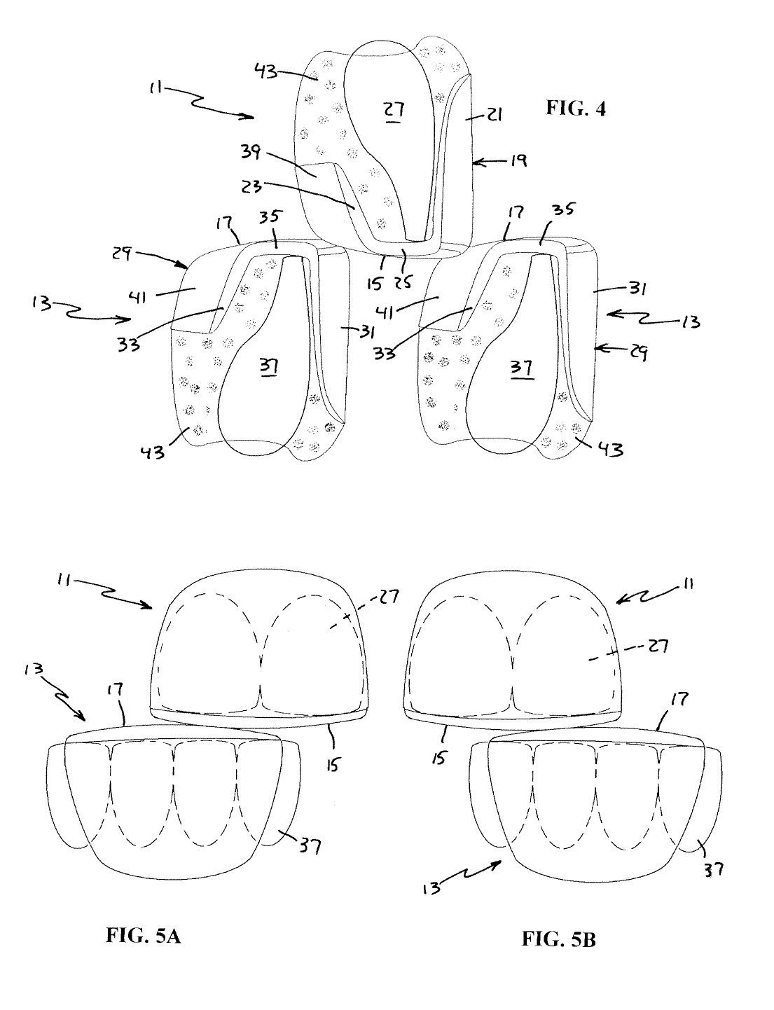

FIG. 4 is a schematic side elevational view of the maxillary appliance 11 and mandibular appliance 13 of FIG. 3, showing the extreme protrusive and retrusive positions of the mandibular appliance relative to the maxillary appliance. Throughout this protrusive/retrusive range of motion, the confronting T-shaped platforms 15, 17 of the two appliances remain in tangential contact with each other.

FIGS. 5A and 5B are schematic front elevational views of the maxillary appliance 11 and mandibular appliance 13 of FIG. 3, showing the extreme excursive positions of the mandibular appliance relative to the maxillary appliance. Throughout this excursive range of motion, the confronting platforms of the two appliances remain in tangential contact with each other.

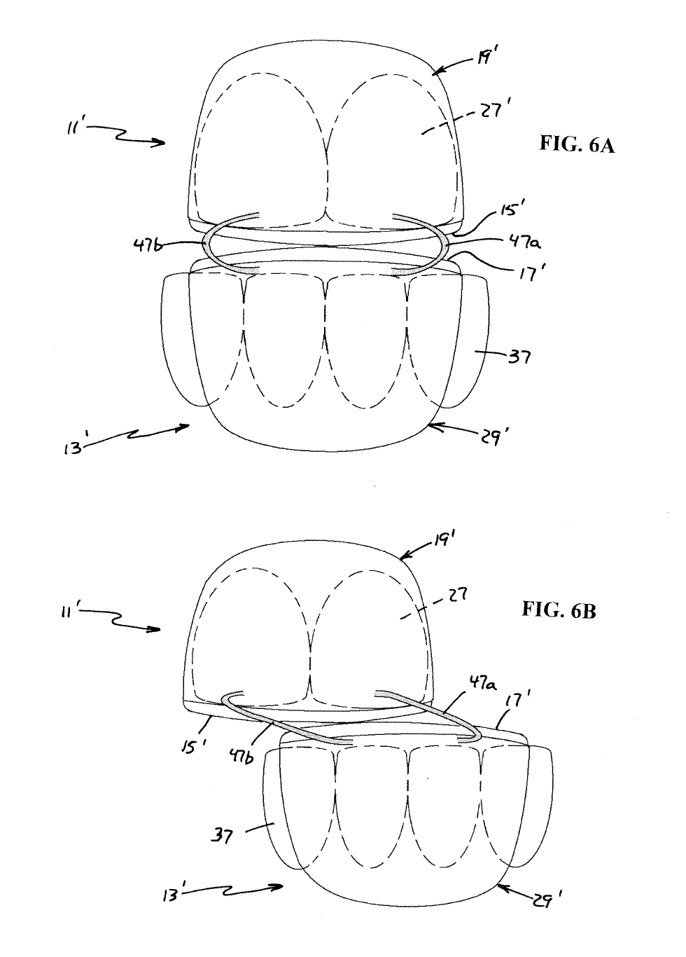

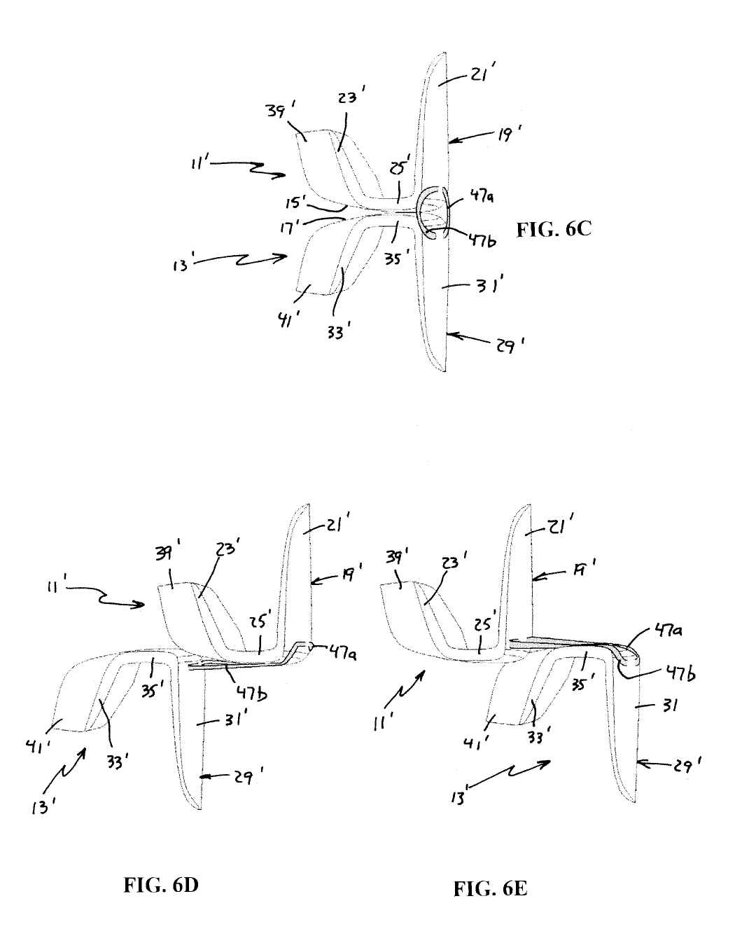

FIGS. 6A-6E depict an alternative embodiment of the invention, in which the maxillary appliance 11′ is loosely connected to the mandibular appliance 13′ by two elastic leashes 47a, 47b. In these figures, the various components are identified using the same numerals as in the prior figures, but with an accompanying apostrophe. These two leashes both extend from the front wall 21′ of the maxillary appliance to the front wall 31′ of the mandibular appliance. The leashes function to provide and retain an ideal relationship of the two appliances relative to each other, thereby facilitating their convenient and efficient installation on the subject's incisors. The leashes also minimize the risk of the appliances being swallowed and aspirated.

The leashes 47a, 47b are sized and configured to allow the full range of unrestricted mandible movement, including excursive movement and protrusive/retrusive movement. These movements are depicted in FIGS. 6B, 6D and 6E. The leashes can be made from suitable materials such as rubber or silicone. They are secured to the appliances 11′ and 13′ using a suitable dental adhesive such as cyanoacrylate. Alternatively, the tooling that is used to make the appliances can be modified to make the leashes an integral part of the appliances.

It should be appreciated from the foregoing description that the present invention provides an improved method and apparatus for use in diagnosing temporomandibular disorders. The apparatus includes separate maxillary and mandibular appliances, which are sized and configured to receive and be retained on the subject's incisors. The two appliances both include generally flat, T-shaped platforms that face each other in a confronting relationship for all possible mandibular movements, including excursive movement and protrusive/retrusive movement. When properly fitted on a subject's opposing incisors, the two appliances effectively prevent contact between the opposing upper and lower teeth, including the posterior and canine teeth, and thereby reduce the intensity of clenching of the temporalis muscles. The apparatus thereby can be used to provide an inexpensive way for determining whether an intraoral discluder of this kind will be effective in relieving tension headaches, common migraine headaches, or other temporomandibular disorders.

Although the invention has been described in detail with reference to the presently preferred embodiments, those of ordinary skill in the art will appreciate that various modifications can be made without departing from the invention. Accordingly, the invention is defined only by the following claims.

I claim:

1. An apparatus for use in diagnosing a temporomandibular disorder, comprising:
a maxillary appliance including a tray that defines a channel sized to receive and be retained on a subject's maxillary incisors; and
a mandibular appliance including a tray that defines a channel sized to receive and be retained on the subject's mandibular incisors;
wherein the maxillary and mandibular appliances both further include platforms arranged in confronting relationship with each other when the appliances are retained on the subject's respective maxillary and mandibular incisors;
and wherein the platforms of the maxillary and mandibular appliances are sized and configured to ensure that they confront each other for all possible mandibular positions and to prevent contact between opposing upper and lower teeth, including the posterior and canine teeth, thereby reducing the intensity of clenching of the subject's temporalis muscles.

2. The apparatus as defined in claim 1, wherein each of the maxillary and mandibular appliances further includes a quantity of adaptable material disposed within the channel of its tray, to conformably adapt to the shape of the incisors and assist in retaining the appliance on the incisors.

3. The apparatus as defined in claim 2, wherein the adaptable material is a curable material.

4. The apparatus as defined in claim 3, and further comprising an approximating device for temporarily fixing the maxillary and mandibular appliances in a prescribed relationship relative to each other while the maxillary and mandibular appliances are disposed within the subject's mouth and the adaptable material of the maxillary and mandibular appliances cures.

5. The apparatus as defined in claim 4, wherein the approximating device comprises a strip of adhesive tape that can be adhered to, and removed from, the maxillary and mandibular appliances.

6. The apparatus as defined in claim 1, wherein the confronting surfaces of the platforms of the maxillary and mandibular appliances each are substantially smooth and convex, such that they contact each other substantially tangentially for all possible mandibular positions.

7. The apparatus as defined in claim 6, wherein the confronting surfaces of the platforms of the maxillary and mandibular appliances each are generally T-shaped, each having a main section aligned with the incisors and a posterior section projecting in a posterior direction from a mid-portion of the main section.

8. The apparatus as defined in claim 1, wherein:
the maxillary tray includes a front wall, a rear wall, and an interconnecting bottom wall that define the channel sized to receive and be retained on the subject's maxillary incisors;
the mandibular tray includes a front wall, a rear wall, and an interconnecting top wall that define the channel sized to receive and be retained on the subject's mandibular incisors; and
the apparatus further includes one or more leashes connecting the maxillary appliance to the mandibular appliance.

9. The apparatus as defined in claim 8, wherein each of the one or more leashes is an elastic band connected at its ends to the maxillary tray and the mandibular tray.

10. The apparatus as defined in claim 8, wherein the one or more leashes include two elastic bands, each connected at one end to the front wall of the maxillary tray and at its opposite end to the front wall of the mandibular tray.

11. An apparatus for use in diagnosing a temporomandibular disorder, comprising:
a maxillary appliance including a tray that includes a rear wall, a front wall, and a bottom wall interconnecting the rear and front walls, such walls together defining a channel sized to receive and be retained on a subject's maxillary incisors;
a mandibular appliance including a tray that includes rear wall, a front wall, and a top wall interconnecting the rear and front walls, such walls together defining a channel sized to receive and be retained on the subject's mandibular incisors; and
a quantity of curable adaptable material disposed within the channels of the maxillary tray and the mandibular tray, to conformably adapt to the shape of the associated incisors and assist in retaining the appliances on the incisors;
wherein the maxillary and mandibular appliances both further include platforms arranged in confronting relationship with each other when the appliances are retained on the subject's respective maxillary and mandibular incisors;
wherein the confronting surfaces of the platforms of the maxillary and mandibular appliances are substantially smooth and convex, such that they contact each other substantially tangentially for all possible mandibular movements and positions;
and wherein the platforms of the maxillary and mandibular appliances are sized and configured to ensure that they confront each other for all possible mandibular positions and to prevent contact between opposing upper and lower teeth, including the posterior and canine teeth, thereby reducing the intensity of clenching of the subject's temporalis muscles.

12. The apparatus as defined in claim 11, and further comprising an approximating device for temporarily fixing the maxillary and mandibular appliances in a prescribed relationship relative to each other while the maxillary and mandibular appliances are disposed within the subject's mouth and the adaptable material of the maxillary and mandibular appliances cures.

13. The apparatus as defined in claim 12, wherein the approximating device comprises a strip of adhesive tape that can be adhered to, and removed from, the maxillary and mandibular appliances.

14. The apparatus as defined in claim 11, wherein the confronting surfaces of the platform of the maxillary and mandibular appliances each are generally T-shaped, each having a main section aligned with the incisors and a posterior section projecting in a posterior direction from a mid-portion of the main section.

15. The apparatus as defined in claim 11, and further comprising one or more leashes connecting the maxillary appliance to the mandibular appliance.

16. The apparatus as defined in claim 15, wherein each of the one or more leashes is an elastic band connected at one end to the front wall of the maxillary tray and at its opposite end to the front wall of the mandibular tray.

17. A method for diagnosing temporomandibular disorders, comprising the steps of:
providing a maxillary appliance that includes a tray having a channel, a curable material disposed within the tray's channel, and a platform located on the side of the tray opposite the channel;
providing a mandibular appliance that includes a tray having a channel, a curable material disposed within the tray's channel, and a platform located on the side of the tray opposite the channel;
placing the maxillary and mandibular appliances within a subject's mouth, with the maxillary appliance overlaying the maxillary incisors and the mandibular appliance overlaying the mandibular incisors, wherein the platforms of the maxillary and mandibular appliances are arranged in confronting relationship with each other when the appliances are retained on the subject's respective maxillary and mandibular incisors;
temporarily fixing the maxillary and mandibular appliances in a prescribed relationship relative to each other, with their respective platforms contacting each other, while the curable material of the maxillary and mandibular appliances cures in a substantially conforming relationship with the respective maxillary and mandibular incisors; and
releasing the maxillary and mandibular appliances from each other after the curable material has cured;
wherein the platforms of the maxillary and mandibular appliances are sized and configured to ensure that they confront each other for all possible mandibular positions and to prevent contact between opposing upper and lower teeth, including the posterior and canine teeth, thereby reducing the intensity of clenching of the subject's temporalis muscles.

18. The method as defined in claim 17, wherein the step of temporarily fixing includes a step of temporarily applying an approximating device to the maxillary and mandibular appliances.

19. The method as defined in claim 18, wherein the approximating device used in the step of temporarily applying is a strip of adhesive tape that can be adhered to, and removed from, the maxillary and mandibular appliances.

20. The method as defined in claim 17, wherein the prescribed relationship of the maxillary and mandibular appliances used in the step of releasably fixing is provided by centering the subject's mandible, with the maxillary and mandibular incisors aligned with each other.
